(19) United States
(12) Patent Application Publication
(10) Pub. No.: US 2025/0152075 A1
(43) Pub. Date: May 15, 2025

(54) CROSS-SESSION ALIGNMENT OF NEURAL RECORDINGS USING SENSORY TASKS

(71) Applicant: UNIVERSITY OF SOUTHERN CALIFORNIA, Los Angeles, CA (US)

(72) Inventors: Maryam Shanechi, Los Angeles, CA (US); Omid Ghasem Sani, Los Angeles, CA (US)

(73) Assignee: UNIVERSITY OF SOUTHERN CALIFORNIA, Los Angeles, CA (US)

(21) Appl. No.: 18/946,641

(22) Filed: Nov. 13, 2024

Related U.S. Application Data
(60) Provisional application No. 63/598,469, filed on Nov. 13, 2023.

(51) Int. Cl. A61B 5/372; A61B 5/378; A61B 5/38; G16H 50/20

(52) U.S. Cl. CPC A61B 5/372; A61B 5/378; A61B 5/38; G16H 50/20

(57) ABSTRACT

A new paradigm of methods and systems can enable cross-session normalization of neural recordings using sensory tasks. In this paradigm, in addition to the primary task being studied, each neural recording session also includes short sensory tasks that evoke event-related potentials (ERPs) from the low-level sensory processing of the brain. Since low-level sensory processing in the brain is expected to undergo minimal plasticity, changes in low-level sensory ERPs can be largely attributed to changes in the recording setup. The new paradigm involves collection of data from sensory task in each recording session and using that collected data to align the neural recordings of different sessions with each other. The aligned neural recordings can then be used within the system, for example in a brain-computer interface system to track mental states or to track the response to interventions such as pharmacological or neurostimulation interventions over time in a given individual.

### CROSS-REFERENCE TO RELATED APPLICATIONS

This application is based upon and claims the benefit of and priority to U.S. Provisional Patent Application No. 63/598,469 entitled “CROSS-SESSION ALIGNMENT OF NEURAL RECORDINGS USING SENSORY TASKS,” filed on Nov. 13, 2023, the entire content of which is incorporated by reference herein.

### STATEMENT AS TO FEDERALLY SPONSORED RESEARCH

This invention was made with government support under contract number NIH R01MH123770 awarded by the National Institute of Health (NIH). The government has certain rights in the invention.

### FIELD

The present disclosure generally relates to systems, devices, and methods for cross-session normalization of neural recordings, for example those obtained by electroencephalogram (EEG), using sensory tasks.

### BACKGROUND

The subject matter discussed in the background section should not be assumed to be prior art merely as a result of its mention in the background section. Similarly, a problem mentioned in the background section or associated with the subject matter of the background section should not be assumed to have been previously recognized in the prior art. The subject matter in the background section merely represents different approaches, which in and of themselves may be inventions.

Non-invasive EEG recordings can be obtained from healthy participants, which gives them a unique advantage over intracranial recording methods for studying brain functions. However, EEG recording setups are temporary in nature, and basic recording characteristics such as electrode impedance and location may vary significantly across recording sessions. These non-neural cross-session variations can mask real variations in brain signals themselves and thus can confound studies that require multi-session sampling of an individual's neural/brain signals, for example to study the encoding of mood or pain or mental state variations in neural signals over days.

To enable such investigations and otherwise use of neural recordings across sessions, a new paradigm that enables cross-session normalization of EEG is desirable.

### BRIEF DESCRIPTION OF THE DRAWINGS

The subject matter of the present disclosure is particularly pointed out and distinctly claimed in the concluding portion of the specification. A more complete understanding of the present disclosure, however, may best be obtained by referring to the following detailed description and claims in connection with the following drawings. While the drawings illustrate various embodiments employing the principles described herein, the drawings do not limit the scope of the claims.

FIG. 1 illustrates a cross-session alignment approach where a series of sensory tasks are added to each recording session to collect neural data that will be used to align recordings across sessions, in accordance with various embodiments.

FIG. 2 illustrates steady-state visually evoked potential (SSVEP) data with 10 Hz in the left column and 15 Hz in the right column, in accordance with various embodiments.

FIG. 3 illustrates variability of SSVEP data across sessions, in accordance with various embodiments.

FIG. 4 illustrates linear mapping learned only using 10 Hz or 15 Hz data, in accordance with various embodiments.

FIG. 5 illustrates the SSVEP for the first and last 40% of the sensory data, in accordance with various embodiments.

FIG. 6 illustrates after applying a mapping trained based on the first 40% of the data, cross-session alignment in the last 40% of the data also substantially increases, suggesting generalization across time within a session, in accordance with various embodiments.

FIG. 7 illustrates linear mapping learned only using 10 Hz or 15 Hz data, in accordance with various embodiments.

FIG. 8 illustrates a system for cross-session normalization of brain recordings using sensory tasks alongside a primary task of interest, in accordance with various embodiments.

FIG. 9 illustrates a method for cross-session normalization of brain recordings using sensory tasks alongside a primary task of interest, in accordance with various embodiments.

FIG. 10 illustrates a method for cross-session normalization of brain recordings using sensory tasks alongside a primary task of interest, in accordance with various embodiments.

### DETAILED DESCRIPTION

The following detailed description of various embodiments herein refers to the accompanying drawings, which show various embodiments by way of illustration. While these various embodiments are described in sufficient detail to enable those skilled in the art to practice the disclosure, it should be understood that other embodiments may be realized and that changes may be made without departing from the scope of the disclosure. Thus, the detailed description herein is presented for purposes of illustration only and not of limitation. Furthermore, any reference to singular includes plural embodiments, and any reference to more than one component or step may include a singular embodiment or step. Also, any reference to attached, fixed, connected, or the like may include permanent, removable, temporary, partial, full or any other possible attachment option. Additionally, any reference to without contact (or similar phrases) may also include reduced contact or minimal contact. It should also be understood that unless specifically stated otherwise, references to “a,” “an” or “the” may include one or more than one and that reference to an item in the singular may also include the item in the plural. Further, all ranges may include upper and lower values and all ranges and ratio limits disclosed herein may be combined.

Disclosed herein is a new paradigm that enables cross-session normalization of EEG using sensory tasks. A key idea developed herein is to use sensory tasks, which are expected to have stable neural response over time as an anchor to normalize neural recordings across sessions. Sensory tasks are those that evoke a response from the brain based on some sensory (e.g., visual or auditory) stimuli. Importantly, sensory tasks evoke responses that are a reflection of the low level immediate sensory processing in the brain as opposed to higher level cognitive functions that may undergo plasticity over time.

In this paradigm, in addition to the primary task being studied, each EEG recording session also includes short sensory tasks that evoke event-related potentials (ERPs) from the low-level sensory processing of the brain. Since low-level sensory processing in the brain is expected to undergo minimal plasticity, changes in low-level sensory ERPs can be largely attributed to changes in the recording setup. Therefore, by the systems and methods disclosed herein, EEG recordings can be normalized such that recordings from different sessions yield similar ERPs for low-level sensory tasks.

The aligned neural recordings obtained using this paradigm can be used for any purpose within the system, for example they can be used in a brain-computer interface system to track brain or mental states over time in a given individual or they can be used to track the response to interventions such as pharmacological or neurostimulation interventions over time in a given individual.

One candidate for visual sensory tasks to use in this paradigm is a steady-state visually evoked potential (SSVEP), which is the response of the brain to visual stimuli that flash on and off with a fixed frequency. SSVEP is however not the only option for the sensory task that could be used for cross-session alignment. Other examples include auditory evoked potential (AEP) and other visually evoked potentials (VEP).

This idea was validated herein by collecting unparalleled multi-session EEG datasets with 20-40 recordings from each participant each collected on a different day. In each recording, a steady-state visually evoked potential (SSVEP) task was included as a low-level sensory task. It was demonstrated that SSVEP responses can be used to learn mappings of the EEG sensor space, which transform EEG in a way that yields matching SSVEP responses from recordings across different sessions. Moreover, EEG sensor space mappings can be learned that can normalize all recording sessions to a comparable sensor space, resulting in similar SSVEPs across all sessions. These results demonstrate the effectiveness of the novel paradigm for cross-session normalization of EEG recordings using sensory tasks disclosed herein. This approach may enable future works to utilize EEG in longitudinal studies of brain functions in individuals over time, in ways that were previously considered impractical with EEG. For example, this approach can be used to track mental states such as mood, depression severity, anxiety severity, or pain over time (e.g., days) based on neural recordings, or it can be used to track an individual's response to pharmacological interventions or drugs over time (e.g., days).

The approach for cross-session alignment of neural recordings disclosed herein relies on adding a short segment to each experimental session where a sensory task is performed by the participant. Data can then be used from the sensory task section of the recordings to build mathematical transformations or mappings that can be applied to data from any given recording session to make it aligned with another recording session. The mappings are thus learned based on the sensory task section in a recording session but are applicable to the whole recording session.

### Methods

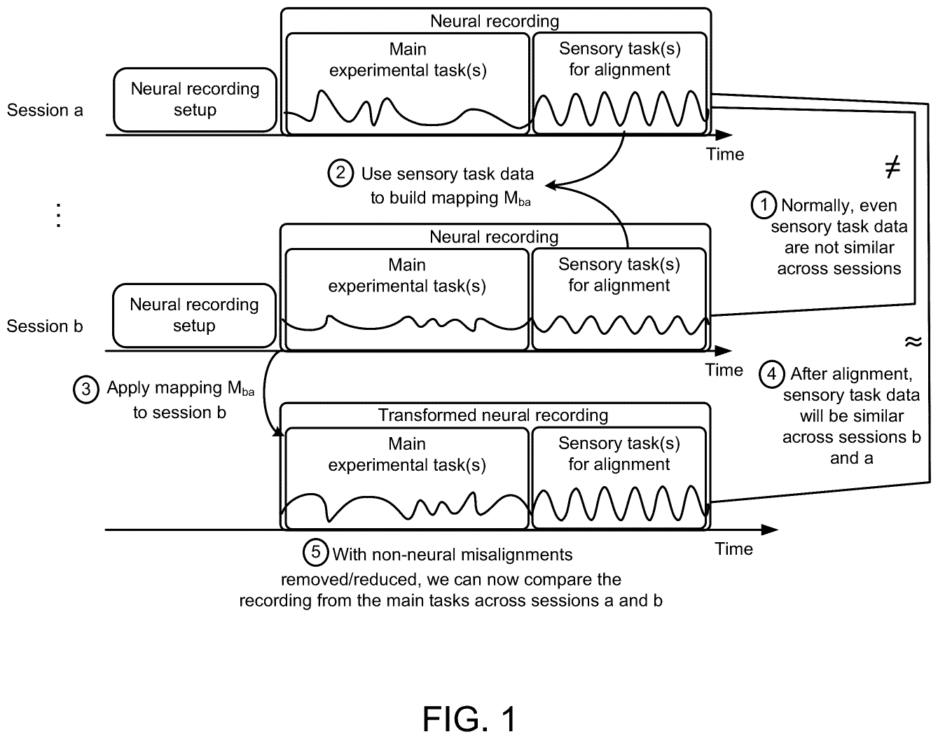

In the cross-session alignment approach disclosed herein, a series of sensory tasks are added to each recording session to collect neural data that will be used to align recordings across sessions (FIG. 1). Briefly, the logic, method steps, and outcomes are as follows (FIG. 1): 1) Brain responses to basic sensory tasks are not expected to have major differences across recording sessions since these responses involve basic non-cognitive processing. However, due to non-neural differences across recordings, such as changes in the recording setup, for example how the EEG cap is placed, even the response to these basic sensory tasks has significant differences across neural recordings. 2) the data recorded are used during the sensory task to learn a mathematical mapping, denoted by Mab, that transform the sensory data from a source session a to look as similar as possible to the sensory data from session b. 3) The learned mapping can then be applied to the full recording of the source session a, including the main tasks of interest in the recording. 4) After the mapping is applied, the neural data from the sensory task would be much more similar across recording sessions a and b, indicating a reduced level of non-neural differences between the two recordings. 5) With non-neural differences accounted for after the alignment, the data from the complete recording, including the main experimental tasks of interest can more effectively be compared and modeled across recording sessions.

### Experimental Data Collection

To experimentally validate the cross-session alignment approach disclosed herein, two series of recordings were collected from two participants. The first and second participant participated in 36 and 22 recording sessions, respectively, with each recording session being on a different day over the course of 1-3 months. A Geodesic EEG System 400 (Magstim EGI, Inc.) was used with 128 channel saline-based sensor nets to record EEG during each session. The participants sat in front of a computer display that guided them through the experimental session. The distance from the participants' eyes from the display was approximately 60 cm. After setting up the EEG cap, the recording channels were inspected both in terms of their impedance and expected time domain characteristics such as visible alpha oscillation when eyes were closed and EMG noise visibility when jaws were clenched. Any irregularities were noted and any channels that were deemed too noisy and could not be fixed by adding additional saline to be excluded from analyses were listed. Across all recording sessions, 4±3 (mean±std) and at most 11 channels were marked as noisy. These noisy channels were interpolated in each recording as the average of surrounding normal channels. Throughout the recordings, aside from rest periods in between experiments, the participants were asked to not move and avoid blinking to the extent possible.

The experimental protocol consisted of three phases, as follows: (1) Participants looked at the center of the display on a fixation point with their eyes open for 1 minute and then relaxed with their eyes closed for 1 minute. This sequence was repeated twice, adding up to 4 minutes of eyes open/closed baseline neural recording to be used in other experiments that are not the subject of this report; (2) The participants were provided with a series of psychometric questionnaires on the display and were asked to respond to them. Again, this phase of the experimental protocol is not subject of this report; and (3) The sensory task phase of the experimental protocol, explained in the next section.

### The Sensory Task

Participants performed a sensory task that comprised looking at the display while a stimulus (the letter ‘A’) was flashed with a fixed frequency on the screen. This type of flashing stimuli elicits a response from the brain that is known as the steady-state visually evoked potential (SSVEP). The key character of the SSVEP brain response is that the same frequency with which the stimuli flashes on the screen has dominant components in the recorded neural responses and these responses are largest in magnitude towards the back of the head where the occipital lope, which contains the visual cortex, is located. In the protocol disclosed herein, the stimuli were shown with a 10 Hz frequency for 30 seconds, then the frequency was immediately switched to 15 Hz shown for another 30 seconds, followed with 10 s rest period with no stimuli. This sequence was repeated 5 times, adding up to a total of 5 minutes of SSVEP recordings, divided up equally between 10 Hz and 15 Hz stimuli. The SSVEP protocol was implemented using a previously developed open-source web-based SSVEP stimulation interface called QuickSSVEP. The interface stores the timestamp of every stimulus on or off event. In addition, for a more precise log of these events, they were also recorded via the EGI system in sync with the EEG by placing a photodiode in front of the display where the stimuli were shown.

### Data Analyses

Custom python code was used to implement all analyses. The EGI system recoded the EEG with a 1000 Hz sampling rate and the same sampling rate was kept for all analyses. The following preprocessing filters were applied to the recorded EEG data. First, a high pass filter was applied with a cut of frequency of 0.1 Hz to remove any slow drifts. Second, notch filters at 60 Hz and its harmonics (120 Hz, etc.) were applied to remove the line noise. Third, the 4±3 (mean±std) channels that were marked as noisy were interpolated in each recording session linearly based on surrounding normal channels. Data loading and preprocessing was done using the open-source MNE library.

SSVEP is the response that is evoked from the brain by a visual stimulus repeating at a fixed frequency, hence the name steady state visually evoked potential. Similar to other event related potentials (ERPs), one can estimate increasingly accurate estimates of the SSVEP by collecting instances of brain recordings that are time locked to the event, and then averaging over these instances. To do this, 400 ms windows centered around all the times at which the visual stimulus turned on were taken, and averages were taken over these 400 ms windows to get the SSVEP. This was done separately for the 10 Hz and the 15 Hz stimuli, resulting in two SSVEP signals. The averaging is done per channel, yielding one 400 ms multi-channel SSVEP signal per frequency. To fully disambiguate this SSVEP computation, it was clarified that in the end, for a given frequency, the computed SSVEP is a matrix E with n_{ch} rows and T columns, where n_{ch} is the number of EEG recording channels and T=400 is the number of data samples in a 400 ms window. To refer to the SSVEP for a given frequency f, the notation E^{(f)} was used, and to refer to the SSVEP for a given recording session a, the subscript Eₐ was used.

To learn a mapping M_{ba} to align a source session b to a target session a, the mapping that minimizes the sum of squares error loss was found:

$$L = {\vert {M}_{b a} ( {E}_{b} ) - {E}_{a} \vert}_{F} \qquad ( 1 )$$

where | |_{F} denotes the sum of squares of all the elements in the matrix. For the class of linear mapping, M_{ba}(E_{b}) is the matrix multiplication M_{ba}E_{b} and the closed-form ordinary least-squares (OLS) solution to the above optimization is

$${M}_{b a} = {E}_{b} {{E}_{a}^{T} ( {E}_{a} {E}_{a}^{T} )}^{- 1} . \qquad ( 2 )$$

For the linear solution, ridge regularization can also be easily added to the optimization, where the closed form solution is:

$${M}_{b a} = {E}_{b} {{E}_{a}^{T} ( {E}_{a} {E}_{a}^{T} + λ {I}_{{n}_{c h}} )}^{- 1} . \qquad ( 3 )$$

where Iₙ_{_{ch}} denotes the n_{ch} by n_{ch} unit matrix and λ is the scalar regularization hyperparameter that when larger will discourage the capacity of the mapping to be fully used, hence reducing overfitting.

A separate mapping can be learned for each stimulus condition (e.g., each SSVEP frequency) by simply including only data from that condition in fitting the mapping. Alternatively, the data can be concatenated from multiple or all conditions horizontally, which can be used to fit the mapping to get one mapping that works for all conditions. It was always specified what conditions were used in learning the mapping for each result.

In principle, this cross-session alignment approach does not depend on a particular mapping method, so while OLS and ridge regression are considered herein, other mappings can also be considered. Examples of other linear mappings that can be considered for M_{ba} include reduced rank regression, lasso regression, and canonical correlation analyses. Examples of nonlinear mappings that can be considered for M_{ba} include support vector regression (SVR) and nonlinear neural networks. A key goal regardless of the method can be to avoid overfitting in the learned mapping. Also, in addition to the objective function in equation (1), other objective functions can also be used for this optimization to find a mapping. Also, nonlinear mappings and any mapping function is possible, such as neural networks, nonlinear regressions, and support vector regressions.

Once the mapping is learned, the mapping can be applied to any part of the data from the source session b to obtain the transformed data for that part of the session. For a linear mapping M_{ba} session, this can be done by a matrix multiplication:

$${E}_{b}^{′} = {M}_{b a} {E}_{b} \qquad ( 4 )$$

where E_{b}′, denoted the transformed data from session b.

One can interpret equation (4) as follows: the mapped SSVEP signal for the i-th channel is a linear combination of the SSVEP signal across all channels where the weights are given by the i-th row of M_{ba}. Note that the same mapping is applied to all time samples (columns of E_{b}), so the mapping is time invariant.

While in equation (4) the letter E is used to denote data, the same transformation can be applied to any part of the data from session b, and not just the sensory task data.

To evaluate the quality of the mapping, the error can be computed between the transformed SSVEP from the source session b with the original SSVEP from the target session a, that is:

$${\vert {M}_{b a} {E}_{b} - {E}_{a} \vert}_{F} . \qquad ( 5 )$$

As another metric, the correlation coefficient (CC) was computed between the E_{b} and Eₐ for each channel (row) and these CC values were averaged across the dimensions.

### Results

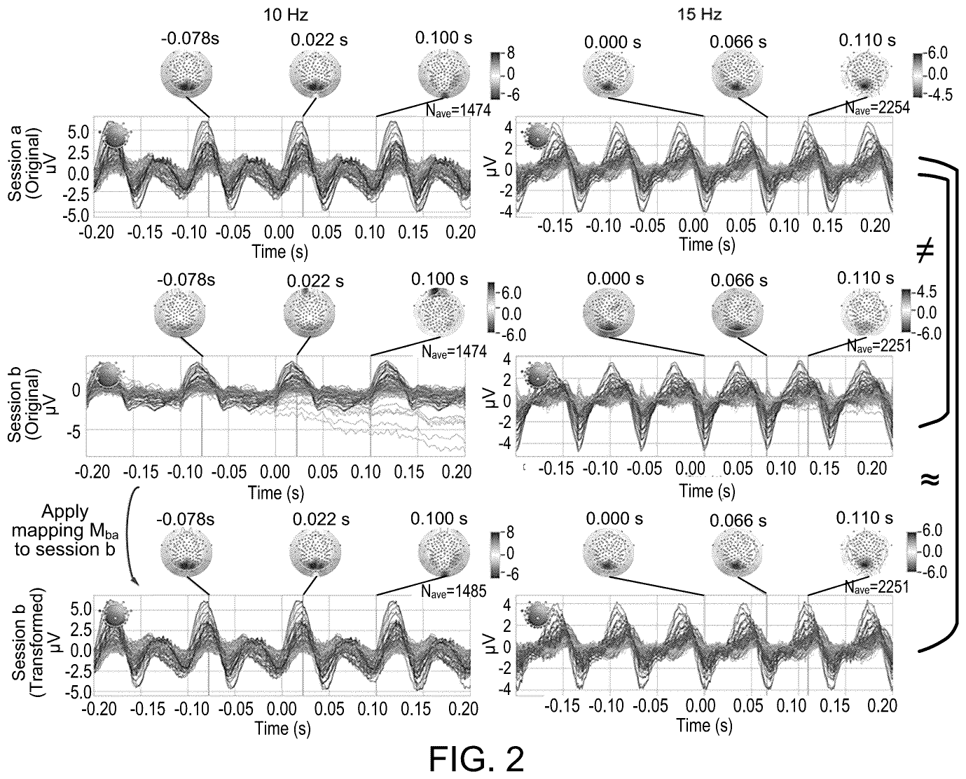

In various embodiments, recorded neural responses had the expected characteristics and demonstrated substantial cross-session variability. To validate the data collection and preprocessing, the computed SSVEP responses were inspected and it was confirmed that they exhibit the key expected characteristics of these signals (FIG. 2). Specifically, recorded SSVEP in all sessions 1) was periodic with the same period as that of the stimuli both for 10 Hz and 15 Hz stimuli, 2) had larger magnitude towards the back of the head where the visual cortex resides. These characteristics confirm the validity of the recording setup and stimulus delivery.

The differences of the recorded SSVEP across recording sessions were then visually inspected. Despite having the same participant, experimental protocol, recording system, and data processing, different recording sessions had substantial differences in the shape of the periodic signals of difference channels, their magnitude, and their apparent lag relative to the stimulus on event (FIG. 2). Next, the cross-session differences were quantified.

Referring now to FIG. 2, the SSVEP data with 10 Hz in the left column and 15 Hz in the right column is illustrated, in accordance with various embodiments. The SSVEP data for session a (row 1) and b (rows 2-3), are shown for session b both before (row 2) and after (row 3) the mapping that aligns it to session a. Each line shows the SSVEP for one channel over a 400 ms window centered around the time a stimulus flashes on the display, for a channel towards the back of the head. The SSVEP value across all recording channels for 3 times during the window is shown on three circular topoplot for 3 times during the window.

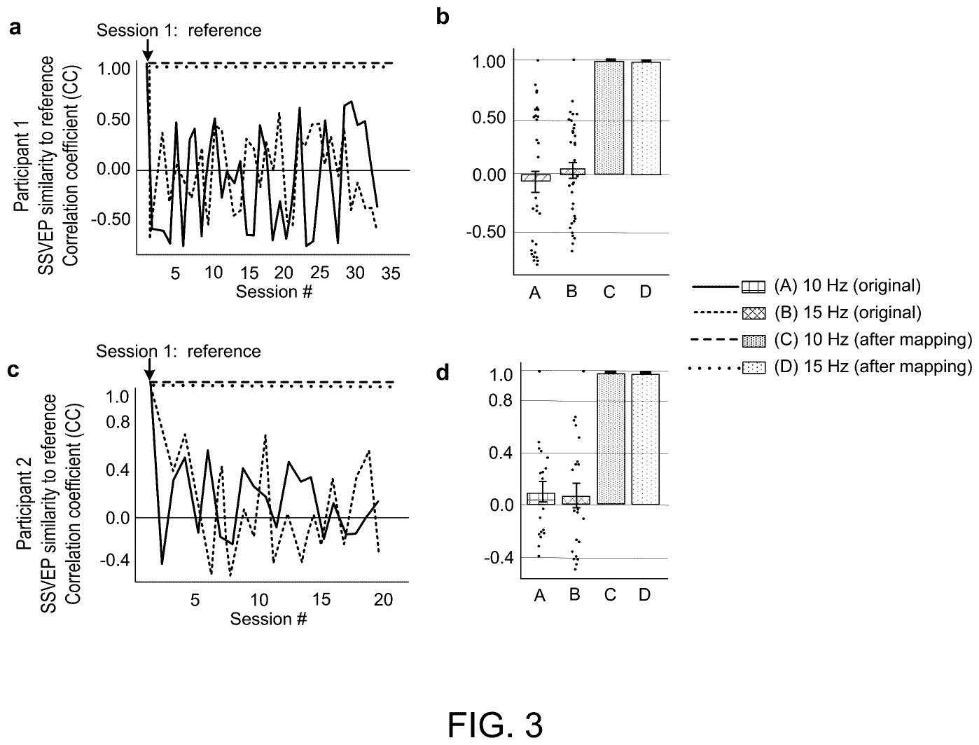

To quantify cross-session differences in the neural recordings, the correlation coefficient (CC) was computed between the SSVEP of each channel for each session with that of the same channel from the first recording session (i.e., the reference session). The CC was then averaged across recording channels to get one final CC value for each recording to quantify its similarity to the first recording session (FIG. 3, lines A,B). The results show substantial misalignment between all sessions from the first session, for both participants and stimuli frequencies (FIG. 3, lines A,B). Given that basic sensory processing in the brain is not expected to experience substantial neural plasticity across sessions, most of the observed cross-session misalignment of EEG can be attributed to non-neural sources, which can confound or mask real neural differences across sessions. This result demonstrates the critical need for a method to mitigate the cross-session misalignment of EEG.

In various embodiments, linear mapping had the capacity to align SSVEP across recording sessions. It was then investigated whether linear mappings have the capacity to align neural recordings from one session onto another. To do so, as explained in the methods described further herein, a linear mapping was fitted for each recording sessions such that the SSVEP after the mapping was as close as possible to the SSVEP from the first recording session (i.e., the reference session). Then, both SSVEP frequencies (10 Hz and 15 Hz) were concatenated in leaning the mapping, so a single mapping was learned for both stimulus frequencies. And the same mapping was applied over time as explained in methods, so the mapping was time invariant. It was found that even this simple time invariant mapping could align the SSVEP data from every session to be very similar to the SSVEP data from the reference session, in both participants (FIG. 3). These results suggest that linear mappings can have the capacity to align neural recordings across sessions.

Referring now to FIG. 3, variability of SSVEP data across sessions is significant, but a linear mapping has the capacity to closely align sessions. In FIG. 3(a), the similarity, as quantified by correlation coefficient (CC), between the first and every other session is shown for 10 Hz and 15 Hz SSVEP, both before (A,B) and after (C,D) the alignment mapping is applied, in accordance with various embodiments. The results are for participant 1. In FIG. 3(b), the same information from FIG. 3(a) is shown as a bar plot, with individual points for each session shown as dots, and the whiskers showing the SEM. In FIGS. 3(c) and 3(d), the same information as FIGS. 3(a) and 3(b) is shown for participant 2.

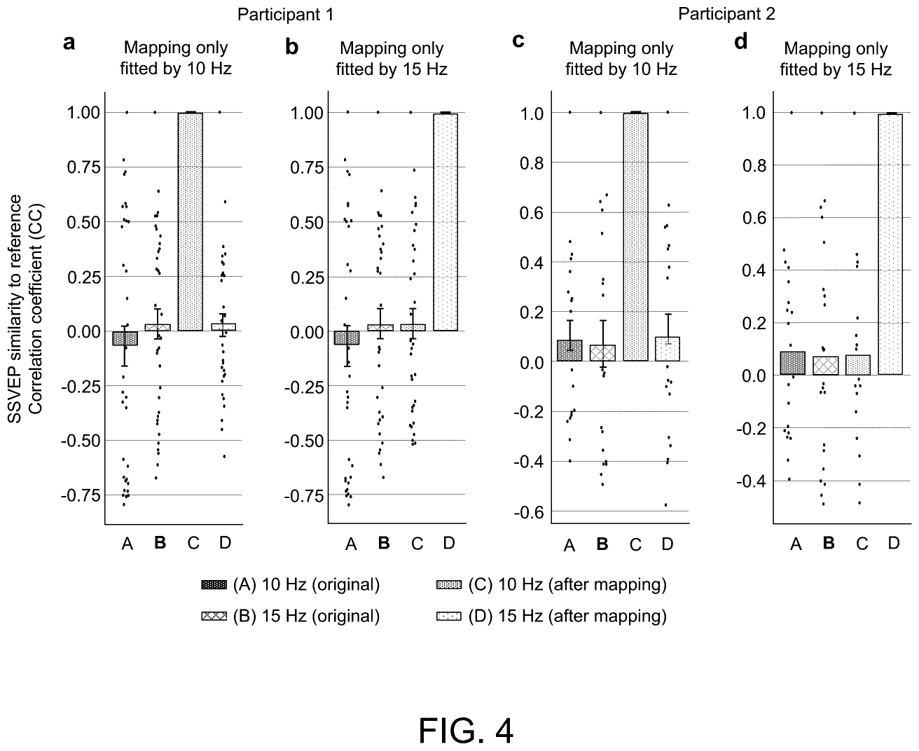

In various embodiments, as previously described herein, it was found that learning a single mapping by combining the SSVEP for both conditions (10 Hz and 15 Hz) was effective in aligning both conditions across sessions (FIG. 3). This analysis was then repeated, but this time separate mappings were fitted for each SSVEP condition: one mapping was learned only using the 10 Hz SSVEP data (FIG. 4a,c) and another only using the 15 Hz SSVEP data (FIGS. 4b,d). Interestingly, the mapping learned from each condition could align that same condition across sessions (FIG. 4) but the combined mapping was more effective for the other frequency (FIG. 3). This result suggests that using multiple sensory task conditions to learn a unified mapping may lead to increased r-performance. Alternatively, or in addition, the improved generalizability for combined conditions may also suggest that non-neural cross-session variabilities may be nonlinear.

Referring now to FIG. 4, results with linear mapping learned only using 10 Hz or 15 Hz data are illustrated, in accordance with various embodiments. FIG. 4(a) is similar to FIG. 3b but showing the results when only the 10 Hz SSVEP is used to learn the alignment mappings. FIG. 4(b) is the same as FIG. 4(a), showing the results for when only the 15 Hz data is used to learn the alignment mappings. FIGS. 4c-d are the same as FIGS. 4a-b, shown for participant 2.

In various embodiments, temporal cross-validation confirmed substantial increase in cross-session alignment of neural data. As discussed previously herein, all the sensory task data was used from two sessions to build a mapping from one session to the other. Here, a temporal cross-validation was performed to confirm that the increase in alignment after the mapping in prior sections is not simply a reflection of overfitting in learning the mapping. The first 40% of the sensory task data was taken from each session as the training data to learn the mapping and the last 40% as the test data to evaluate the mapping. This ensured that there was no overlap between the data used to learn the mapping and the data used to quantify its effectiveness in aligning cross-session neural data.

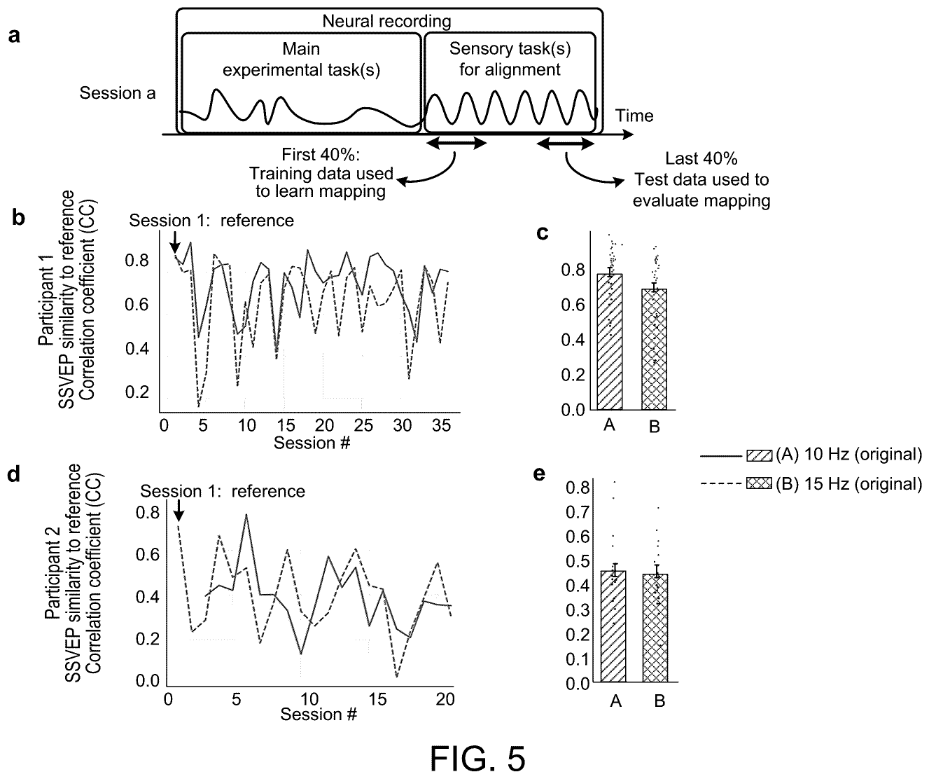

The recording sessions in this dataset included roughly 5 minutes of data from the sensory tasks, resulting in around 2 minutes of training data in cross-validation analyses. For 10 Hz and 15 Hz stimuli this adds up to roughly 1200 and 1800 events, respectively, that can be averaged over to estimate the SSVEP. Thus, there is also inherent noise in the estimation of the SSVEP that would be reduced with longer data. Due to this SSVEP estimation error that depends on amount of data in a specific experiment, perfect alignment wasn't expected, even between the training and test SSVEP data of the same recording session. To separate the effect of this SSVEP estimation error (that is simply due to amount of data in a specific experiment) from cross-session misalignment, the correlation was computed between the training and test data of each session as an upper bound for the maximum alignment that could be expected between the test data across sessions (FIG. 5). The mean of this within-session alignment was computed across all sessions of each subject as a normalization factor and the cross-session results were divided up by this value.

Referring now to FIG. 5, due to finite data, the first and last 40% of the sensory data lead to somewhat different SSVEP, providing a ceiling for cross-session alignment across data segments in this dataset is illustrated, in accordance with various embodiments. In FIG. 5(a), the schema showing how only for each session, the first 40% of the data and the last 40% are taken and training and test data, respectively. In FIG. 5(b) the correlation coefficient between the first and last 40% of the sensory task data in each session for participant 1 is illustrated, in accordance with various embodiments. FIG. 5(c) provides the same information shown as a bar plot, with the same notation as in FIG. 3b. FIGS. 5d-e are the same as FIGS. 5b-c, shown for participant 2.

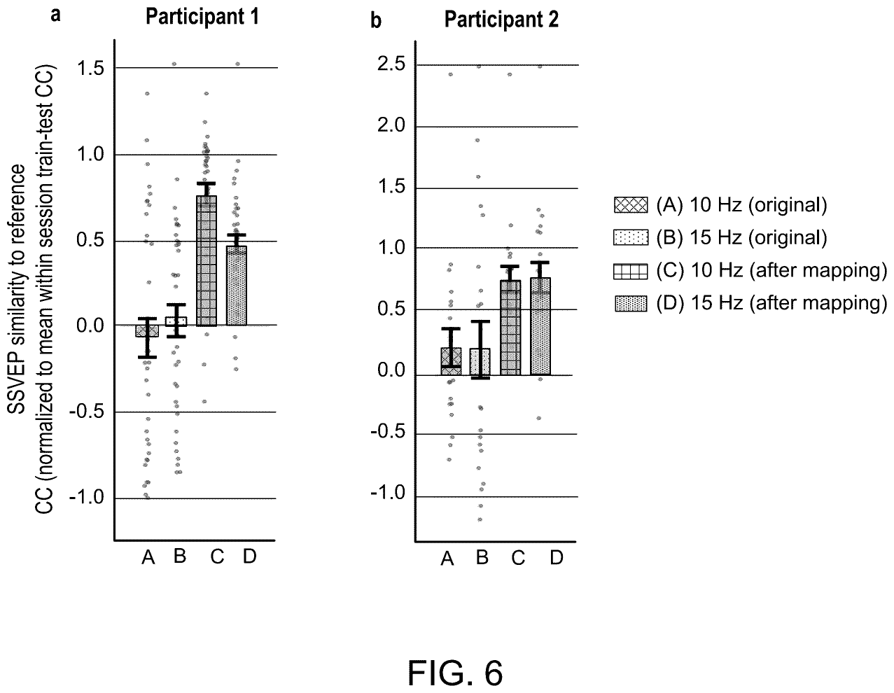

The mapping analyses was repeated, but only used the training section of the data (i.e., the first 40%) from each session to learn the mapping and only used the test section of the data (i.e., the last 40%) to evaluate the post-mapping alignment of the neural recordings across sessions. First, the analyses in FIG. 3 were repeated, but only the training section of the data from both stimulus frequencies (both 10 Hz and 15 Hz) were used to fit the mappings from one session to another. It was found that the learned mapping substantially increased the alignment of the unseen test data across sessions for both 10 Hz and 15 Hz stimuli (FIG. 6). Specifically, the correlation coefficient (CC) of the test data across sessions reached around 50% to 75% of the average CC between the training and test data of the same session (FIG. 6). This suggests that the approach disclosed herein indeed substantially increases the alignment between neural recordings beyond what might be attributable to overfitting.

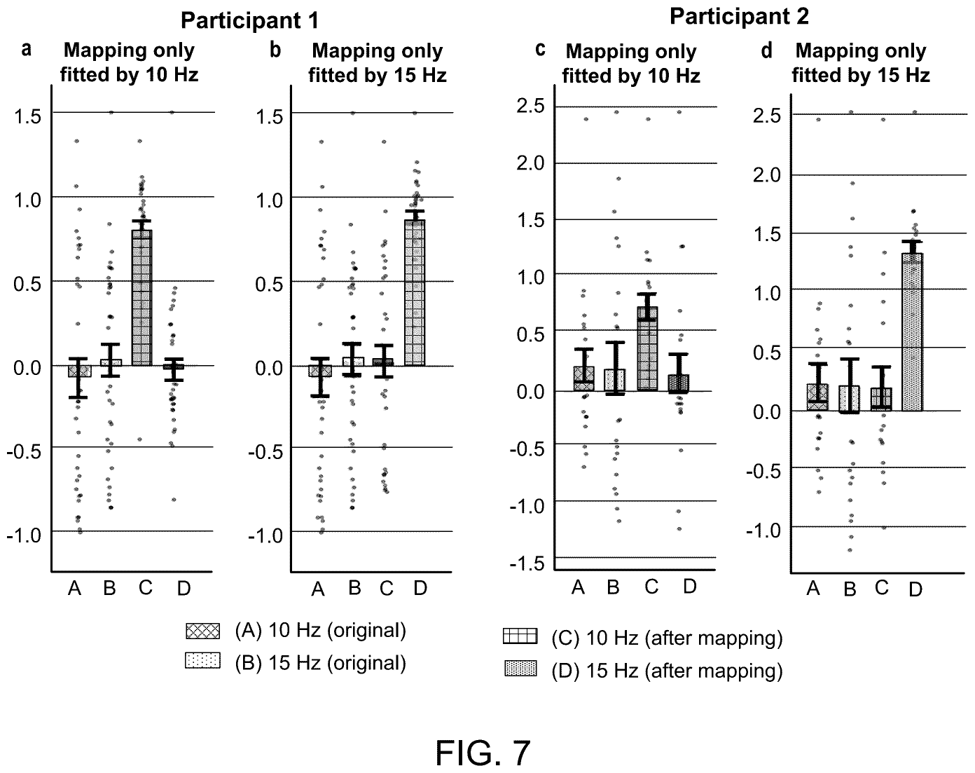

Next, it was investigated whether mappings learned using a combination of stimulus frequencies would outperform one that is learned only with data from one stimulus frequency (e.g., only from only 10 Hz SSVEP) in terms of aligning the data for the other stimulus frequency in the test data. To so, a separate mapping using the training data was used for each stimulus frequency and these mappings were evaluated on the test data of both frequencies (FIG. 7). It was found that similar to FIGS. 3 and 4, the mappings learned for combined conditions (FIG. 6) indeed outperformed the mappings learned for a given frequency (FIG. 7). Given the lack of overlap between training and test data, overfitting does not explain this result. Thus, these results further suggest that cross-session variability of neural signals are nonlinear.

Referring now to FIG. 6, after applying a mapping trained based on the first 40% of the data, cross-session alignment in the last 40% of the data also substantially increases, suggesting generalization across time within a session, in accordance with various embodiments. Same as in FIG. 3, FIG. 6 shows the similarity of the SSVEP to the reference session (i.e., session 1) in the test set (last 40% of the sensory task data).

Referring now to FIG. 7, linear mapping learned only using 10 Hz or 15 Hz data performed well for the same frequency, in accordance with various embodiments. Same as in FIG. 4, FIG. 7 shows the similarity of the SSVEP to the reference session (i.e., session 1) in the test set (last 40% of the sensory task data). Again, the mapping learned using combined frequencies (FIG. 5) was more accurate in aligning both frequencies simultaneously than mappings learned using individual frequencies (FIG. 7).

### Systems and Methods

Disclosed herein is a developed and experimentally validated method for aligning neural recording across sessions. A key idea was to add a sensory task to the experimental protocol of any given task of interest, so that the data from the sensory task can be used to learn a mapping that would align recording sessions with each other. The mapping is learned such that the neural response to the sensory task, which is not expected to have plasticity from session to session, would be consistent across aligned sessions.

It was first confirmed that even linear mappings have the capacity to align recordings across sessions (FIG. 3). It was next shown that using multiple stimulus conditions when learning the mapping can improve generalizability compared with using just one stimulus condition (FIG. 4). Both results were then confirmed in a temporal cross-validation (FIG. 5) showing that they hold even if separate parts of the sensory task data is used to learn the mapping and validate it (FIGS. 6 and 7). These results demonstrate experimental validation of a core idea, namely that sensory tasks can be used to align cross-session neural recordings, in accordance with various embodiments.

Adding more conditions (e.g., additional SSVEP frequencies) in the experimental protocol may be beneficial for obtaining richer data that could enable more generalizable alignment as described herein. In designing the experimental protocol and choosing the conditions for the sensory task, particular focus can be given to covering more frequencies, especially those needed by the downstream tasks. Here, frequencies 10 Hz and 15 Hz were used in the sensory task, so the learned mappings are likely most accurate around these frequencies. One could include more than two stimulus frequencies to expand the frequencies that are accurately aligned. To apply a series of learned mappings for different frequencies to the same data, one could filter the data into different frequency ranges using a filter bank, apply the closest mapping to the filtered signal in each bank, and then add the results of the mappings back up across the banks.

A visual sensory task, i.e., SSVEP, was used in this work to demonstrate the potential of the approach disclosed herein. However, in principle any sensory task with low level brain processing can be used instead of or in conjunction with SSVEP to learn cross-session alignment mappings. For example, auditory evoked potentials (AEP) have similar characteristics to visually evoked potentials such as SSVEP. AEP can thus be instead of SSVEP or in addition to SSVEP during the sensory task to potentially enhance the learned mapping. Given that visual and auditory sensory processing happen in different brain regions, they produce the largest neural response in different recording channels and thus using them both could lead to recording higher quality brain responses from more channels, which could result in a more accurate mappings in general.

Here, the validation dataset consisted of a relatively short sensory task that was 5 mins in length, adding up to around 1490 and 2250 events (i.e., instances of stimulus turning on) for the 10 Hz and 15 Hz stimuli, respectively. Higher frequencies yielded more events per unit time, but in general longer data with more trials is expected to converge toward increasingly more stable estimates of event related potentials, which could result in even more accurate learning of the mapping. In the data discussed herein, it was quantified how much the mapping quality was affected by data size by quantifying the alignment of the training and test sections of the same session (FIG. 5). The alignment was successful even with this amount of data but this analysis suggests that longer than 5 minutes of sensory task data collection can result in even more accurate mappings and alignment.

Here, ridge regression was used to learn the mappings from one session to another. The results suggest that linear mappings have the capacity to align neural data across sessions almost perfectly when the complete sensory task data for a session is learned to fit the mapping (FIG. 3). It is also possible that using other models for the transformation could improve the results further. Other linear methods such as reduced-rank regression or canonical correlation analyses, or nonlinear methods such as support vector regression (SVR) or multilayer perceptron (MLPs) or neural networks can be used in this novel paradigm described herein and lead to an improved mapping capacity. The best approach for learning the transformation may depend on datasets. Ultimately, for any approach, ideally having longer data to learn the mapping would be beneficial.

To reduce overfitting, one key idea used herein is to fit the mapping based on single trial data instead of trial averaged data. In this approach, E was defined as single trial windows of data concatenated horizontally, so the matrix E in this case would still have n_{ch} rows, but will have T×N columns, where N is the number of trials. This approach has the downside that single trial data would be noisier, but has the benefit that single trial has a lot more samples. A middle ground is to sub-sample single trials, average across these subsets to get less noisy averaged SSVEP signals, and then learn the mapping based on the horizontal concatenation of these subset samples. One can use single trials or any subset averaging to find better mapping performance for a given data.

A system, apparatus and/or method for cross-session normalization of brain recordings using sensory tasks alongside a primary task of interest is disclosed herein to perform the AI and machine learning algorithms.

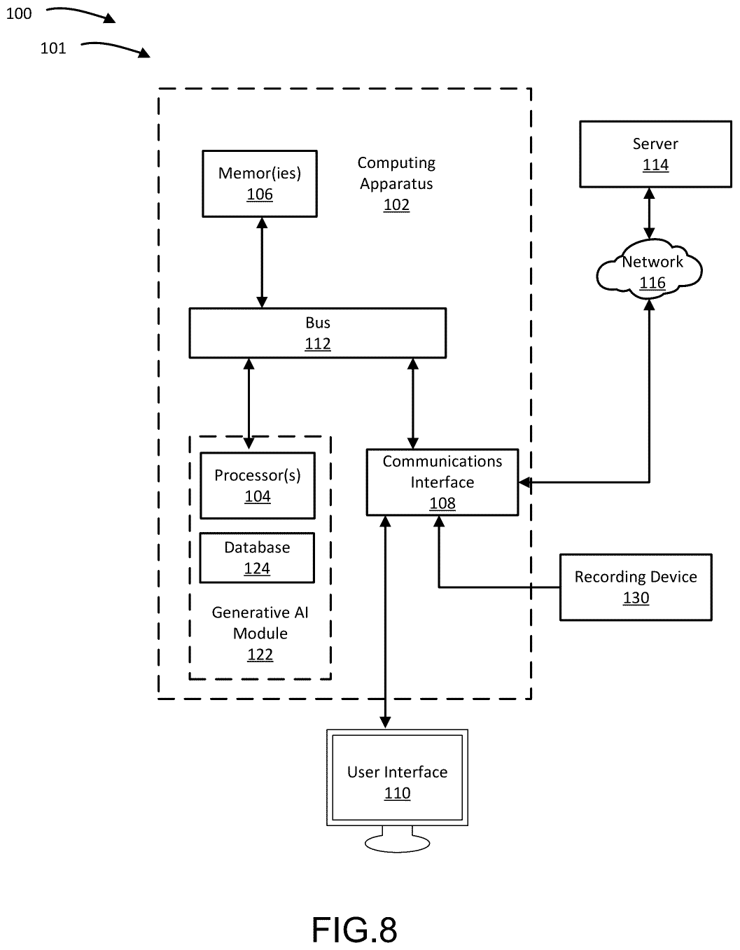

Referring now to FIG. 8, a system 100 for cross-session normalization of brain recordings using sensory tasks alongside a primary task of interest is illustrated, in accordance with various embodiments. The system 100 (e.g., a computing system) may include a computing apparatus 102. The computing apparatus 102 may include one or more processors 104, one or more memories 106 and/or one or more buses 112 and/or other mechanisms for communicating between the one or more processors 104. The system 100 may be a cloud computing system including processors, servers, storage, databases, networking, software, analytics, and/or intelligence accessed or performed over or using the Internet (“the cloud”). The one or more processors 104 may be implemented as a single processor or as multiple processors. The one or more processors 104 may execute instructions stored in the memory 106 to implement the applications and/or detection of the system 100.

The one or more processors 104 may be coupled to the memory 106. The memory 106 may include one or more of a Random Access Memory (RAM) or other volatile or non-volatile memory. The memory 106 may be a non-transitory memory or a data storage device, such as a hard disk drive, a solid-state disk drive, a hybrid disk drive, or other appropriate data storage, and may further store machine-readable instructions, which may be loaded and executed by the one or more processors 104.

The memory 106 may include one or more of random-access memory (“RAM”), static memory, cache, flash memory and any other suitable type of storage device or computer readable storage medium, which is used for storing instructions to be executed by the one or more processors 104. The storage device or the computer readable storage medium may be a read only memory (“ROM”), flash memory, and/or memory card, that may be coupled to a bus 112 or other communication mechanism. The storage device may be a mass storage device, such as a magnetic disk, optical disk, and/or flash disk that may be directly or indirectly, temporarily, or semi-permanently coupled to the bus 112 or other communication mechanism and be electrically coupled to some or all the other components within the system 100 including the memory 106, the user interface 110 and/or the communications interface 108 via the bus 112.

The term “computer-readable medium” is used to define any medium that can store and provide instructions and other data to a processor, particularly where the instructions are to be executed by a processor and/or other peripheral of the processing system. Such medium can include non-volatile storage, volatile storage, and transmission media. Non-volatile storage may be embodied on media such as optical or magnetic disks. Storage may be provided locally and in physical proximity to a processor or remotely, typically by use of network connection. Non-volatile storage may be removable from computing system, as in storage or memory cards or sticks that can be easily connected or disconnected from a computer using a standard interface.

The system 100 may include a user interface 110. The user interface 110 may include an input/output device. The input/output device may receive user input, such as a user interface element, hand-held controller that provides tactile/proprioceptive feedback, a button, a dial, a microphone, a keyboard, or a touch screen, and/or provides output, such as a display, a speaker, an audio and/or visual indicator, or a refreshable braille display. The display may be a computer display, a tablet display, a mobile phone display, an augmented reality display or a virtual reality headset. The display may output or provide cross-session normalization of brain recordings.

The user interface 110 may include an input/output device that receives user input, such as a user interface element, a button, a dial, a microphone, a keyboard, or a touch screen, and/or provides output, such as a display, a speaker, headphones, an audio and/or visual indicator, a device that provides tactile/proprioceptive feedback or a refreshable braille display. The speaker may be used to output audio associated with the audio conference and/or the video conference. The user interface 110 may receive user input that may include configuration settings for one or more user preferences, such as a selection of joining an audio conference or a video conference when both options are available, for example.

The system 100 may have a network 116 connected to a server 114. The network 116 may be a local area network (LAN), a wide area network (WAN), a cellular network, the Internet, or combination thereof, that connects, couples and/or otherwise communicates between the various components of the system 100 with the server 114. The server 114 may be a remote computing device or system that includes a memory, a processor and/or a network access device coupled together via a bus. The server 114 may be a computer in a network that is used to provide services, such as accessing files or sharing peripherals, to other computers in the network.

The system 100 may include a communications interface 108, such as a network access device. The communications interface 108 may include a communication port or channel, such as one or more of a Dedicated Short-Range Communication (DSRC) unit, a Wi-Fi unit, a Bluetooth® unit, a radio frequency identification (RFID) tag or reader, or a cellular network unit for accessing a cellular network (such as 3G, 4G or 5G). The communication interface may transmit data to and receive data from the different components.

The server 114 may include a database. A database is any collection of pieces of information that is organized for search and retrieval, such as by a computer, and the database may be organized in tables, schemas, queries, reports, or any other data structures. A database may use any number of database management systems. The information may include real-time information, periodically updated information, or user-inputted information.

In various embodiments, the computing apparatus 102 can include a generative artificial intelligence (“AI”) module 122. The generative AI module 122 can include the one or more processors 104. Stated another way, the generative AI module 122 can be run, or operated by the one or more processors 104. In various embodiments, the generative AI module 122 can perform the steps of the methods claimed herein and output or provide cross-session normalization of brain recordings using sensory tasks alongside a primary task of interest to the user interface 110.

In various embodiments, the generative AI module 122 is configured to decode cross-session normalization of brain recordings from a database 124 as described further herein. In this regard, the generative AI module 122 is configured to update a generative AI model based on the brain signals and data received from the database 124. In various embodiments, the brain signals from the generative AI module 122, as described further herein can include the methods described herein.

In various embodiments, the system 100 further comprises a recording device 130. In various embodiments, the recording device 130 comprises electroencephalogram (EEG) devices or any other electrophysiological recording devices that may be readily apparent to one skilled in the art. For example, the recording device 130 can be configured for recording intracranial EEG or local field potentials and would still be within the scope of this disclosure. Also, the recording device can be a wearable device or an implantable device. In various embodiments, the recording device 130 is electronically coupled (e.g., wirelessly or wired) to the database 124 (e.g., via the communications interface 108). In this regard, after a recording session, brain recordings and/or neural recordings can be stored in the database 124 and utilized by the generative AI module 122 as described further herein.

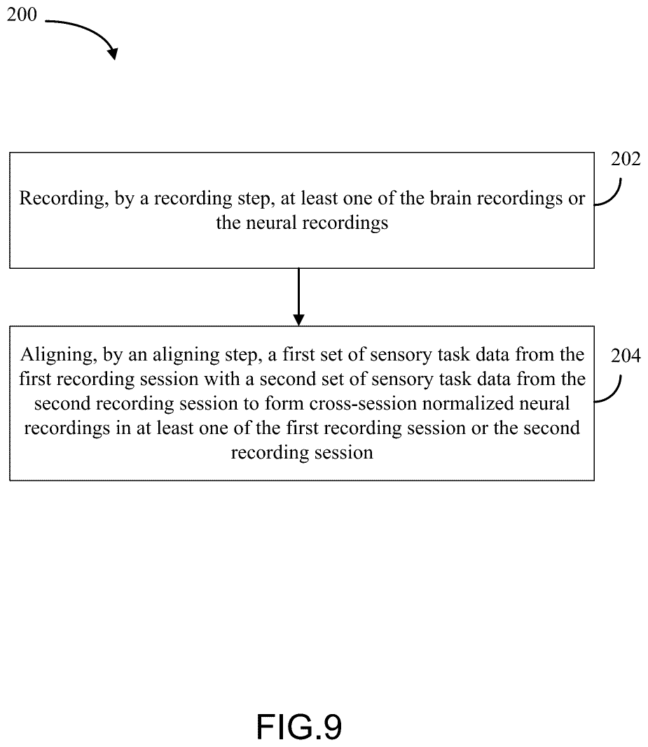

Referring now to FIG. 9, a method 200 (e.g., performable by the system 100 from FIG. 8) for cross-session normalization of at least one of brain recordings or neural recordings is illustrated, in accordance with various embodiments. The method 200 comprises recording, by a recording step, at least one of the brain recordings or the neural recordings (step 202). In various embodiments, the recording step comprises recording, using one or more sensory tasks alongside a primary task of interest, a first recording session of at least one of a first plurality of brain signals or a first plurality of neural signals to generate a first set of the brain recordings or the neural recordings; and recording, using the one or more sensory tasks alongside the primary task of interest, a second recording session of at least one of a second plurality of brain signals or a second plurality of neural signals to generate a second set of the brain recordings or the neural recordings. In various embodiments, the recording step comprises recording a plurality of recording sessions. In various embodiments, any number of recording sessions can be performed in the recording step. The present disclosure is not limited in this regard.

In various embodiments, the method 200 further comprises aligning, by an aligning step, a first set of sensory task data from the first recording session with a second set of sensory task data from the second recording session to form cross-session normalized neural recordings in at least one of the first recording session or the second recording session (step 204).

In various embodiments, the primary task of interest can be any task for which at least one of the brain recordings or the neural recordings are at least one of collected, compared, or combined across multiple recording sessions, the multiple recording sessions including the first recording session and the second recording session.

In various embodiments, the recording the first recording session in step 202 can further comprise obtaining the first set of the brain recordings or the neural recordings using a recording device, the recording device comprising electroencephalogram (EEG) devices or any other electrophysiological recording devices such as those for recording intracranial EEG or local field potentials. In various embodiments, the recording the second recording session in step 202 further comprises obtaining the second set of the brain recordings or the neural recordings using the recording device. In various embodiments, the method 200 further comprising setting up the recording device (e.g., recording device 130 from FIG. 8) again prior to the first recording session and the second recording session, thereby causing mismatches between conditions of recording channels across the first recording session and the second recording session. In various embodiments, the recording device used to obtain the first set of the brain recordings or the neural recordings and the recording device used to obtain the second set of the brain recordings or the neural recordings can be the same device or different devices. The present disclosure is not limited in this regard.

In various embodiments, the one or more sensory tasks are those that show visual stimuli to a user or play auditory stimuli for the user or do both while neural signals are being recorded.

In various embodiments, the recording step in step 202 further comprises collecting a first additional sensory task dataset along with the primary task of interest at least one of before or after the first recording session. In various embodiments, the recording step in step 202 further comprises collecting a second additional sensory task dataset along with the primary task of interest at least one of before or after the second recording session. In various embodiments, the aligning step in step 204 further comprises further aligning, by using the first additional sensory task dataset and the second additional sensory task dataset, the first recording session with the second recording session to form at least one of the cross-session normalized brain recordings or the cross-session normalized neural recordings. In various embodiments, the first additional sensory task dataset and the second additional sensory task dataset each correspond to data collected during performance of an additional sensory task that is a visual task known as a steady-state visually evoked potential (SSVEP), wherein a visual stimulus is shown to the user on a screen while it flashes with a fixed frequency, wherein the visual stimulus optionally comprises an image or a shape. In various embodiments, one or more settings of the SSVEP task or other visually-evoked or auditory-evoked sensory tasks are used, wherein the one or more settings optionally include different stimulus frequencies. In various embodiments, at least one of the SSVEP task or another visually-evoked sensory task or an auditory-evoked sensory task is used. In various embodiments, one or both of a visually-evoked sensory task and an auditory-evoked sensory task is used. In various embodiments, the visual stimuli is a letter that is shown on a display while flashing with a frequency that remains fixed for any set amount of time but then changes to another frequency, alternating between multiple frequencies, and wherein the letter optionally includes a letter “A,” or any other letter. In various embodiments, a number of frequencies presented and a total duration of one of the one or more sensory tasks is expanded depending on how much time is available in each of the first recording session and the second recording session, to collect the first sensory task dataset and the second sensory task dataset, each of which is over some set number of minutes and cover a wide range of frequencies, each presented for some set number of minutes in total.

In various embodiments, the first sensory task dataset from the one or more sensory tasks of the first recording session and the second sensory task dataset from the one or more sensory tasks of the second recording session were each divided into windows around each time the stimuli were provided, with each window being referred to as a trial. In various embodiments, the aligning step further comprises grouping the trial of a given stimuli type from the first recording session and the trial from the given stimuli type during the second recording session together in groups known as conditions, wherein the given stimuli type optionally includes every time the stimuli appeared during a stimulation with a set frequency in the first recording session or the second recording session. In various embodiments, the recording step further comprises recording a plurality of recording sessions, wherein the trial of each condition for each of the plurality of recording sessions are averaged for each time-step to get an event related potential for each recording channel, each condition, and each recording session. In various embodiments, the plurality of recording sessions includes the first recording session, the second recording session, and a third recording session. In various embodiments, a mapping function is learned to make an event related potentials of a given recording session map onto the event related potentials of another reference session from the plurality of recording sessions, wherein the mapping function describes a mathematical operation to combine at least one of a given set of the brain recordings or the neural recordings from the given recording session across all recording channels to get new values for each channel that are more similar than original values of the given recording session to the event related potentials of the given reference session from the plurality of recording sessions.

In various embodiments, the mapping function is learned using linear regression. In various embodiments, the mapping function is learned using other methods, for example regularized ridge regression, support vector regression, a neural network, or a multi-layer neural network. In various embodiments, the mapping function is learned using a subset of SSVEP data, and tested using remaining data to evaluate a generalization of the learned mapping function. In various embodiments, the mapping function is learned using grand averages across the trial of each condition and based on averages taken over subsets of trials in each condition, such that a total number of averaged signals from which the mapping function is learned increases. In various embodiments, the learned mapping function is applied to at least one of the brain recordings or the neural recordings from the primary task of interest to make them more comparable across recording sessions. In various embodiments, the mapping function is learned in a frequency-specific manner, with a separate mapping function being learned for each frequency condition of the one or more sensory tasks. In various embodiments, the mapping function is learned for multiple frequencies, with the same mapping function being learned for each frequency condition of the one or more sensory tasks.

In various embodiments, to apply frequency-specific mappings on neural data, the neural data is decomposed into streams that are filtered in different frequency bands around each stimuli frequency, an associate mapping function is applied to each filtered data stream, and resulting mapping streams are added back.

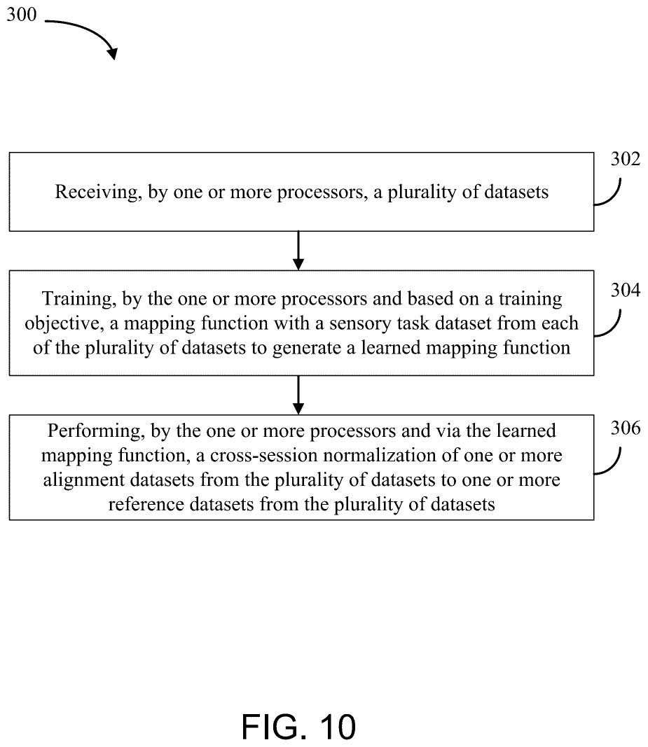

Referring now to FIG. 10, a method 300 (performable by the system 100 from FIG. 8) for cross-session normalization of at least one of brain recordings or neural recordings is illustrated in accordance with various embodiments. In various embodiments, the method 300 comprises: receiving, by one or more processors, a plurality of datasets (step 302). In various embodiments, each of the plurality of datasets corresponds to a recording session using one or more sensory tasks alongside a primary task of interest. In various embodiments, each of the plurality of datasets comprises at least one of brain recordings or neural recordings. In various embodiments, the method 300 further comprises training, by the one or more processors and based on a training objective, a mapping function with a sensory task dataset from each of the plurality of datasets to generate a learned mapping function (step 304). In various embodiments, the method 300 further comprises performing, by the one or more processors and via the learned mapping function, a cross-session normalization of one or more alignment datasets from the plurality of datasets to one or more reference datasets from the plurality of datasets (step 306).

Benefits, other advantages, and solutions to problems have been described herein regarding specific embodiments. Furthermore, the connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements of the disclosure. The scope of the disclosure is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B and C may be present in a single embodiment; for example, A and B, A and C, B and C, or A and B and C. Different cross-hatching is used throughout the figures to denote different parts but not necessarily to denote the same or different materials.

Systems, methods, and apparatus are provided herein. In the detailed description herein, references to “one embodiment,” “an embodiment,” “various embodiments,” etc., indicate that the embodiment described may include a particular feature, structure, or characteristic, but every embodiment may not necessarily include the feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether explicitly described. After reading the description, it will be apparent to one skilled in the relevant art(s) how to implement the disclosure in alternative embodiments.

Furthermore, no element, component, or method step in the present disclosure is intended to be dedicated to the public regardless of whether the element, component, or method step is explicitly recited in the claims. No claim element herein is to be construed under the provisions of 35 U.S.C. 112(f) unless the element is expressly recited using the phrase “means for.” As used herein, the terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus.

Finally, any of the above-described concepts can be used alone or in combination with any or all the other above-described concepts. Although various embodiments have been disclosed and described, one of ordinary skill in this art would recognize that certain modifications would come within the scope of this disclosure. Accordingly, the description is not intended to be exhaustive or to limit the principles described or illustrated herein to any precise form. Many modifications and variations are possible considering the above teaching.

What is claimed is:

1. A method for cross-session normalization of neural recordings, the method comprising:
recording a first set of neural signals during one or more sensory tasks alongside one or more primary tasks of interest to generate a first recording session;
recording a second set of neural signals during the one or more sensory tasks alongside the one or more primary tasks of interest to generate a second recording session; and
aligning, by an aligning step, a first set of sensory task data from the first recording session with a second set of sensory task data from the second recording session to form cross-session normalized neural recordings in at least one of the first recording session or the second recording session.

2. The method of claim 1, wherein the one or more primary tasks of interest comprises a task for which each of the first set of neural signals and the second set of neural signals are at least one of collected, compared, or combined across multiple recording sessions, the multiple recording sessions, wherein:
the first recording session is a reference recording session; and
the second recording session is aligned to the first recording session via the aligning step.

3. The method of claim 2, wherein:
the recording the first recording session further comprises obtaining the first set of neural signals using a recording device, the recording device comprising electroencephalogram (EEG) devices or any other electrophysiological recording devices such as those for recording intracranial EEG or local field potentials, and
the recording the second recording session further comprises obtaining the second set of neural signals using the recording device.

4. The method of claim 3, further comprising setting up the recording device again prior to the first recording session and the second recording session, thereby causing mismatches between conditions of recording channels across the first recording session and the second recording session.

5. The method of claim 4, wherein the one or more sensory tasks are those that show visual stimuli to a user or play auditory stimuli for the user or do both while at least one of the first set of neural signals or the second set of neural signals are being recorded.

6. The method of claim 5, further comprising:
collecting a first additional sensory task dataset along with the one or more primary tasks of interest at least one of before or after the first recording session; and
collecting a second additional sensory task dataset along with the one or more primary tasks of interest at least one of before or after the second recording session, wherein the aligning step further comprises further aligning, by using the first additional sensory task dataset and the second additional sensory task dataset, the first recording session with the second recording session to form the cross-session normalized neural recordings.

7. The method of claim 6, wherein the first additional sensory task dataset and the second additional sensory task dataset each correspond to data collected during performance of an additional sensory task that is at least one of a steady-state visually evoked potential (SSVEP) task or an auditory-evoked potential sensory task, wherein a stimulus is supplied to the user with at least one of a fixed frequency or different frequencies, wherein the stimulus optionally comprises an image or a shape.

8. The method of claim 6, wherein the first additional sensory task dataset and the second additional sensory task dataset each correspond to data collected during performance of an additional sensory task that is an auditory evoked potential (AEP) task, wherein an auditory stimulus is played for the user with at least one of a fixed frequency or different frequencies.

9. The method in any of claim 7 or 8, wherein one or more settings of the additional sensory task are used, wherein the one or more settings optionally include different stimulus frequencies.

10. The method of claim 7, wherein the visual stimuli is at least one of the shape or the letter that is shown on a display while flashing with a frequency that remains fixed for any set amount of time but then changes to another frequency, alternating between multiple frequencies, and wherein if a letter is shown, the letter optionally includes a letter “A” or any other letter.

11. The method in any of claims 7 or 8, wherein a number of frequencies presented and a total duration of one of the one or more sensory tasks is expanded depending on how much time is available in each of the first recording session and the second recording session, to collect a first sensory task dataset and a second sensory task dataset, each of which is over some set number of minutes and cover a wide range of frequencies, each presented for some set number of minutes in total.

12. The method of claim 6, wherein a first sensory task dataset from the one or more sensory tasks of the first recording session and a second sensory task dataset from the one or more sensory tasks of the second recording session are each divided into windows around each time the stimuli is supplied, with each window being referred to as a trial.

13. The method of claim 12 wherein the aligning step further comprises grouping the trial of a given stimuli type from the first recording session and the trial from the given stimuli type during the second recording session together in groups known as conditions, wherein the given stimuli type optionally includes every time the stimuli occurred during a stimulation with a set frequency in the first recording session or the second recording session.

14. The method of claim 13, further comprising recording the multiple recording sessions, wherein the trial of each condition for each of the multiple recording sessions are averaged for each time-step to get an event related potential for each recording channel, each condition, and each recording session, wherein the multiple recording sessions includes the first recording session and the second recording session.

15. The method of claim 14, wherein a mapping function is learned to make an event related potentials of a given recording session map onto the event related potentials of another reference recording session from the first set of neural signals and the second set of neural signals, wherein the mapping function describes a mathematical operation to combine a given set of neural signals across all recording channels from a given recording session to get new values for each channel that are more similar than original values of the given recording session to the event related potentials of the reference recording session.

16. The method of claim 15, wherein the mapping function is learned using linear regression.

17. The method of claim 15, wherein the mapping function is learned using other methods, for example regularized ridge regression, support vector regression, or a multi-layer neural network.

18. The method of claim 15, wherein the mapping function is learned using a subset of sensory task data, and tested using remaining data to evaluate a generalization of the learned mapping function.

19. The method of claim 15, wherein the mapping function is learned using grand averages across trials of each condition and based on averages taken over subsets of trials in each condition, such that a total number of averaged signals from which the mapping function is learned increases.

20. The method of claim 15, wherein the learned mapping function is applied to neural recordings from the one or more primary tasks of interest to make them more comparable across recording sessions.

21. The method of claim 15 wherein the mapping function is learned in a frequency-specific manner, with a separate mapping function being learned for each frequency condition of the one or more sensory tasks.

22. The method of claim 21, wherein to apply frequency-specific mappings on the second set of neural signals, the second set of neural signals are decomposed into streams that are filtered in different frequency bands around each stimuli frequency, an associate mapping function is applied to each filtered data stream, and resulting mapping streams are added back to form the cross-session normalized neural recordings.

23. The method of claim 1, further comprising recording a plurality of neural signal datasets during the one or more sensory tasks alongside the one or more primary tasks of interest, the plurality of neural signal datasets including a first neural signal dataset including the first set of neural signals, a second neural signal dataset including the second set of neural signals, and a third neural signal dataset including a third set of neural signals, wherein the aligning step further comprises one or more alignment datasets from the plurality of neural signal datasets to one or more reference datasets from the plurality of neural signal datasets.

24. A method for cross-session normalization of neural recordings, the method comprising:
receiving, by one or more processors, a plurality of datasets, each of the plurality of datasets corresponding to a recording session using one or more sensory tasks alongside a primary task of interest, each of the plurality of datasets comprising at least one of brain recordings or neural recordings;
training, by the one or more processors and based on a training objective, a mapping function with a sensory task dataset from each of the plurality of datasets to generate a learned mapping function; and
performing, by the one or more processors and via the learned mapping function, a cross-session normalization of one or more alignment datasets from the plurality of datasets to one or more reference datasets from the plurality of datasets.

25. A system for cross-session normalization of at least one of a plurality of brain recordings or a plurality of neural recordings, the system comprising:
one or more processors; and
one or more tangible, non-transitory memories configured to communicate with the one or more processors, the one or more tangible, non-transitory memories having instructions stored thereon that, in response to execution by the one or more processors, cause the one or more processors to perform operations comprising:
performing, by the one or more processors and via a learned mapping function, a cross-session normalization of one or more alignment datasets from a plurality of datasets to one or more reference datasets from the plurality of datasets, wherein the learned mapping function is configured to align a first set of sensory task data from the one or more reference datasets with a second set of sensory task data from the one or more alignment datasets to form cross-session normalized neural recordings.
